(19) United States
(12) Patent Application Publication
(10) Pub. No.: US 2024/0114862 A1
(43) Pub. Date: Apr. 11, 2024

(54) LOSS-OF-FUNCTION TRANSCRIPTION FACTOR-LIKE GENE AND SELF-PROPAGATING FAGOPYRUM PLANT USING SAME

(71) Applicants: Kyoto University, Kyoto (JP); Nissey Delica Corp., Tokyo (JP)

(72) Inventors: Yasuo YASUI, Kyoto (JP); Mariko UENO, Kyoto (JP); Yasumasa KAWATE, Tokyo (JP)

(21) Appl. No.: 18/547,428

(22) PCT Filed: Feb. 21, 2022
(86) PCT No.: PCT/JP2022/006820

§ 371 Date: Aug. 22, 2023

(30) Foreign Application Priority Data
Feb. 22, 2021 (JP) 2021-026492

(51) Int. Cl. A01H 1/06; A01H 1/00; C12N 15/01

(52) U.S. Cl. CPC A01H 1/06; A01H 1/121; C12N 15/01

(57) ABSTRACT

Provided is a loss-of-function transcription factor-like gene and a self-propagating Fagopyrum plant using the same. A self-propagating Fagopyrum plant having both long stamen and long pistil has been bred by: producing S-ELF3-PS1 gene which is a loss-of-function transcription factor-like gene by identifying a transcription factor-like gene formed by a linked gene group that controls lengths of stamen and pistil of a Fagopyrum plant from mapping results of a genomic sequence of the Fagopyrum plant, and allowing the transcription factor-like gene to lose a function by mutagenesis; and mating and cultivating an individual having this S-ELF3-PS1 self-propagation gene.

### TECHNICAL FIELD

The present invention relates to a loss-of-function transcription factor-like gene and a self-propagating Fagopyrum plant using the same, particularly, relates to a self-propagating Fagopyrum plant that is capable of being self-propagating.

### BACKGROUND ART

A region where recombination is extremely suppressed is present in the genome of higher organisms. In the region, a gene group that controls plurality of different traits is capable of forming one gene cluster (supergene), and only a combination of specific traits is observed as a complex adaptive trait in populations and in species.

For example, for the distyly of Fagopyrum plants which are angiosperms, a linked gene group that controls self-incompatibility and lengths of stamen and pistil forms self-incompatibility locus supergene (S-supergene), which leads to the success of high efficiency of outcrossing.

As shown in FIG. 12, species of Fagopyrum plants which are distyly plants include individuals having a “flower type with short pistil and long stamen (short-styled flower)” and individuals having a “flower type with long pistil and short stamen (long-styled flower)”. Since heights of anther and stigma are identical between the short-styled flower and the long-styled flower, pollination efficiency between cross-compatible mates via flower visiting insects is enhanced.

As mentioned above, Fagopyrum plants include the short-styled flower and the long-styled flower, and pollination between cross-compatible mates via flower visiting insects is performed. Therefore, a problem thereof is poor pollination efficiency and a poor yield.

In order to improve yields of distyly plants such as Fagopyrum plants, use of loss-of-function genes has received attention. A conventional technique thereof is as disclosed in Patent Literature 1. No such approach is known about Fagopyrum plants.

### CITATION LIST

### Patent Literature

Patent Literature 1: WO2014/115680

### SUMMARY OF INVENTION

### Technical Problem

An object of the present invention is to provide a loss-of-function transcription factor-like gene and a self-propagating Fagopyrum plant utilizing the same.

S-supergene of Fagopyrum plants has two alleles, S and s haplotypes. The genotype of a short-styled flower individual is S/s, and the genotype of a long-styled flower individual is s/s. The present inventors have successfully identified a transcription factor-like gene (S-locus early flowering 3, S-ELF3) considered as a component of the S-supergene (Yasui et al., 2012) ((Yasui Y, Mori M, Aii J, Abe T, Matsumoto D, et al. (2012) S-LOCUS EARLY FLOWERING 3 Is Exclusively Present in the Genomes of Short-Styled Buckwheat Plants that Exhibit Heteromorphic Self-Incompatibility. PLOS ONE 7 (2): e31264. https://doi.org/10.1371/journal.pone.0031264).

This gene was present only in short-styled flower individuals having S haplotype and absent in long-styled flower individuals having no S haplotype. Hence, S-ELF3 has been presumed to play an important role in controlling a short-styled flower trait, though the role has been unknown.

Accordingly, in order to elucidate a function of the S-ELF3 gene and further breed self-propagating buckwheat that has lost distyly, loss-of-function S-ELF3-PS1 gene encoding the S-ELF3 protein has been developed using EMS. Buckwheat individuals having the S-ELF3-PS1 gene are capable of self-propagating, and self-propagating Fagopyrum plants using this gene have been bred.

### Solution to Problem

A feature of the invention of claim 1 is a loss-of-function transcription factor-like gene produced by: identifying a transcription factor-like gene formed by a linked gene group that controls lengths of stamen and pistil of a Fagopyrum plant from mapping results of a genomic sequence of the Fagopyrum plant; and allowing the transcription factor-like gene thus identified to lose a function by mutagenesis.

A feature of the invention of claim 2 is that in the invention described in claim 1, the transcription factor-like gene is S-ELF3 gene which is present only in a short-styled flower individual having S haplotype and absent in a long-styled flower individual having no S haplotype.

A feature of the invention of claim 3 is that in the invention described in claim 1, the mutagenesis produces S-ELF3-PS1 gene by allowing the transcription factor-like gene to lose a function by ethyl methanesulfonate treatment.

A feature of the invention of claim 4 is that in the invention described in claim 3, in the S-ELF3-PS1 gene, a splicing site of intron 3 corresponding to a base at position 3046 of a genomic sequence of the S-ELF3 gene has been altered from G to A so that a codon at positions 2976 to 2978 has become a new stop codon.

A feature of the invention of claim 5 is a self-propagating Fagopyrum plant bred by: producing S-ELF3-PS1 gene which is a loss-of-function transcription factor-like gene by identifying a transcription factor-like gene formed by a linked gene group that controls lengths of stamen and pistil of a Fagopyrum plant from mapping results of a genomic sequence of the Fagopyrum plant, and allowing the transcription factor-like gene to lose a function by mutagenesis; and mating and cultivating an individual having this S-ELF3-PS1 self-propagation gene.

A feature of the invention of claim 6 is that in the invention described in claim 5, in the mating and the cultivation, the breeding is performed by mating the individual having the S-ELF3-PS1 gene with a long-styled flower individual to separate an individual having long stamen and pistil.

### Advantageous Effects of Invention

According to the present invention, a self-propagating Fagopyrum plant bred by: producing S-ELF3-PS1 gene which is a loss-of-function transcription factor-like gene by identifying a transcription factor-like gene formed by a linked gene group that controls lengths of stamen and pistil of a Fagopyrum plant from mapping results of a genomic sequence of the Fagopyrum plant, and allowing the transcription factor-like gene to lose a function by mutagenesis; and mating and cultivating an individual having this S-ELF3-PS1 self-propagation gene, is obtained. Therefore, advantageous effects are exerted by which a self-propagating Fagopyrum plant that offers a high harvest can be provided.

### BRIEF DESCRIPTION OF DRAWINGS

FIG. 1 is a diagram showing the genomic nucleotide sequence of normal S-ELF3 gene.

FIG. 2 is a diagram showing the genomic nucleotide sequence of normal S-ELF3 gene.

FIG. 3 is a diagram showing the genomic nucleotide sequence of normal S-ELF3 gene.

FIG. 4 is a diagram showing the genomic nucleotide sequence of normal S-ELF3 gene.

FIG. 5 is a diagram showing the genomic nucleotide sequence of normal S-ELF3 gene.

FIG. 6 is a diagram showing the genomic nucleotide sequence of mutant S-ELF3-PS1 gene produced by the present invention.

FIG. 7 is a diagram showing the genomic nucleotide sequence of mutant S-ELF3-PS1 gene produced by the present invention.

FIG. 8 is a diagram showing the genomic nucleotide sequence of mutant S-ELF3-PS1 gene produced by the present invention.

FIG. 9 is a diagram showing the genomic nucleotide sequence of mutant S-ELF3-PS1 gene produced by the present invention.

FIG. 10 is a diagram showing the genomic nucleotide sequence of mutant S-ELF3-PS1 gene produced by the present invention.

FIG. 11 is a photograph showing a Fagopyrum plant produced by the present invention.

FIG. 11 is a photograph illustrating pollination between cross-compatible mates of Fagopyrum plants.

### DESCRIPTION OF EMBODIMENTS

Hereinafter, Examples for carrying out the present invention will be described in detail with reference to the attached drawings.

First, the induction of mutation that allowed S-ELF3 gene, a transcription factor-like gene, to lose a function, and mass breeding were performed.

Induction of Mutation and Mass Breeding

In order to allow S-ELF3 to lose a function, mutagenesis with EMS (ethyl methanesulfonate treatment) was carried out. Approximately 4,600 seeds were treated with a 0.33% EMS (ethyl methanesulfonate) solution for 15 hours and cultivated in Kyoto University Experimental Farm. Three to six seeds were harvested from one individual to obtain 7,490 seeds. The seeds were further treated with 0.6% EMS for 15 hours, and seeds were finally harvested from 2,710 individuals. For seed harvesting, one or two seeds were harvested per individual. The seeds were inoculated and cultivated in Kyoto University Experimental Farm, and seeds were harvested from 3,336 individuals on an individual basis. At the time of cultivation, the leaves of 12 individuals were collectively subjected in equal amounts to DNA extraction to prepare 278 DNA bulks (12 individuals×278 bulks=3,336).

Next, an approach of developing S-ELF3-PS1, a loss-of-function transcription factor-like gene, using mutation detection of the S-ELF3 gene will be described.

Development of S-ELF3-PS1 Using Mutation Detection

PCR primers for S-ELF3 were designed, and PCR amplification was performed with the 278 DNA bulks as templates under the following conditions.

98° C. for 2 min, (98° C. for 10 sec, 58° C. for 5 sec, and 72° C. for 90 sec)×30 cycles, and 72° C. for 5 sec

PCR primers were as follows.

TILL-2NEW_S-ELF3-RH_Fw,
TGGGCTTCCATATTTTTAATCGTC
TILL-2NEW_S-ELF3-RH_Rv,
GTAAGTCCTCAAAAGGGCAAATGA

After PCR, the PCR products were prepared into libraries on a bulk basis using Nextera XT DNA Library Prep kit (Illumina, Inc.), and nucleotide sequence reads were obtained using Hiseq X (Illumina, Inc.). Then, the cleaning of the reads using trimmomatic 0.3.2 and mapping to the reference sequence (nucleotide sequence of S-ELF3) with BWA (Li and Durbin, 2009) (Fast and accurate short read alignment with Burrows-Wheeler transform, Bioinformatics, 25, 1754?1760) were performed.

Then, treatment (BAM conversion, sorting, and mpileup) with samtools (Li et al., 2009) (The Sequence Alignment/Map format and SAMtools, Bioinformatics, 25, 2078-2079), Vcf preparation with VarScan (Koboldt et al., 2009) (VarScan: variant detection in massively parallel sequencing of individual and pooled samples. Bioinformatics (Oxford, England), 25, 2283-2285 PMID: 19542151), and mutation detection with SnpEff (Cingolani et al., 2012) (A program for annotating and predicting the effects of single nucleotide polymorphisms, SnpEff: SNPs in the genome of Drosophila melanogaster strain w1118; iso-2; iso-3) were performed.

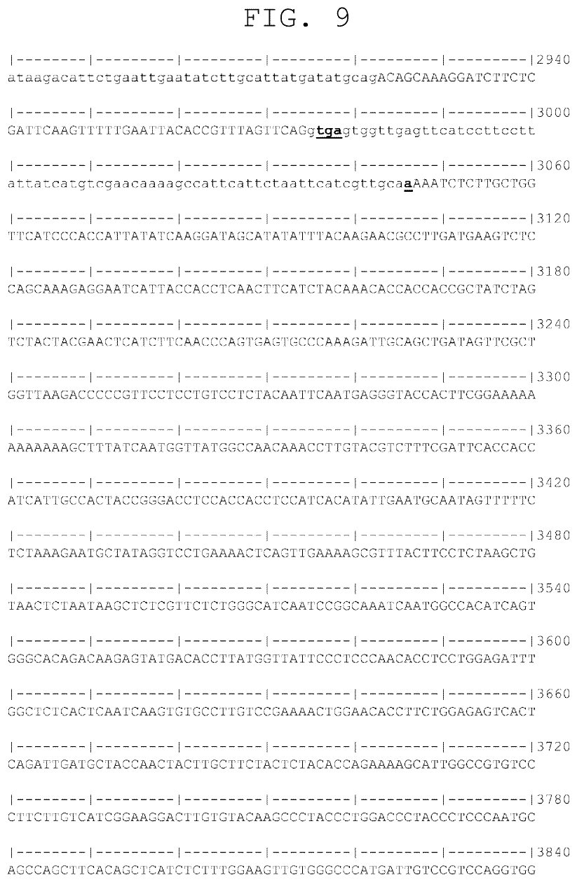

As a result, as shown in boldface and underlines in FIG. 9, S-ELF3-PS1 gene was developed by altering a splicing site of intron 3 corresponding to a base at position 3046 of a genomic sequence of S-ELF3 from G to A.

In the S-ELF3-PS1 gene, as shown in boldface and underlines in FIG. 9, intron 3 remains in mRNA without being spliced. Therefore, a codon at positions 2976 to 2978 becomes a new stop codon. As a result, the function of the S-ELF3-PS1 protein is considered to be lost.

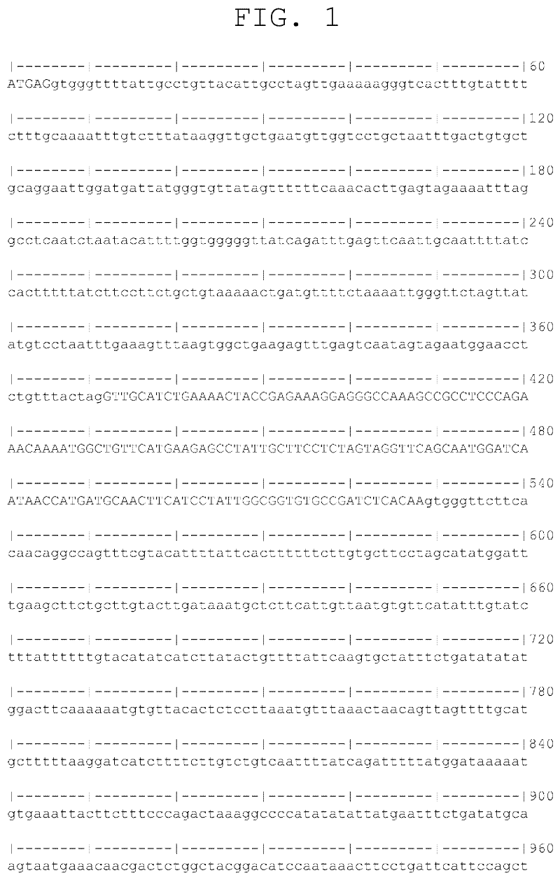

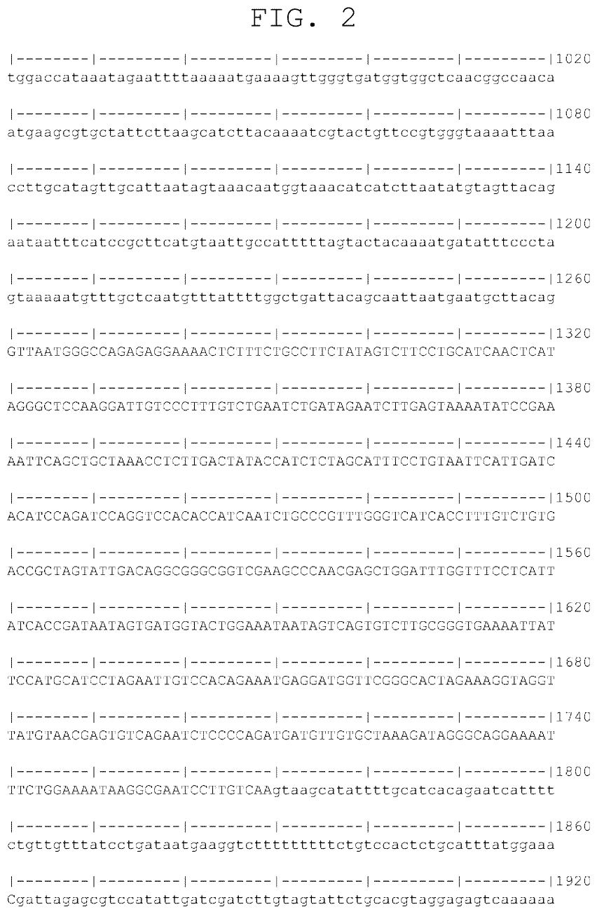

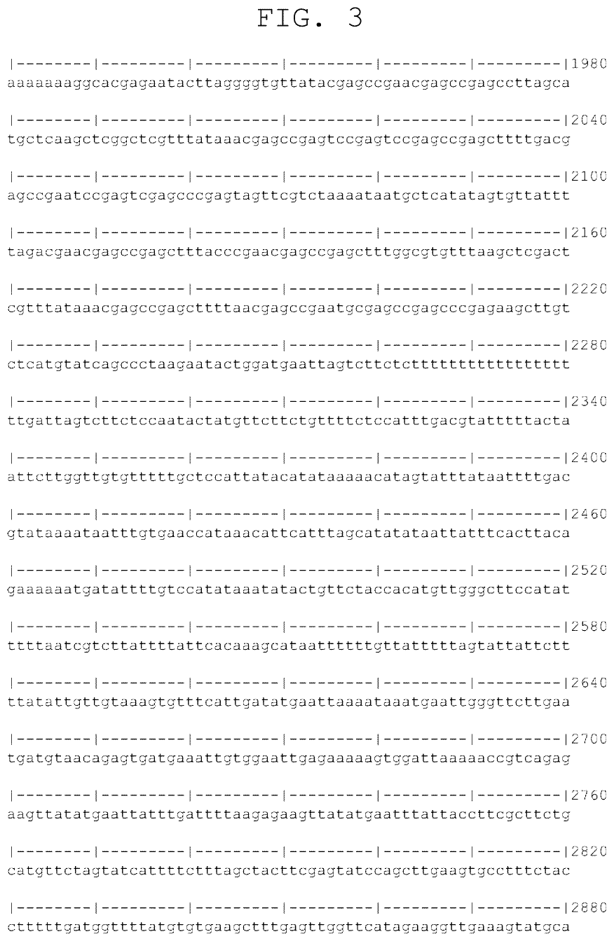

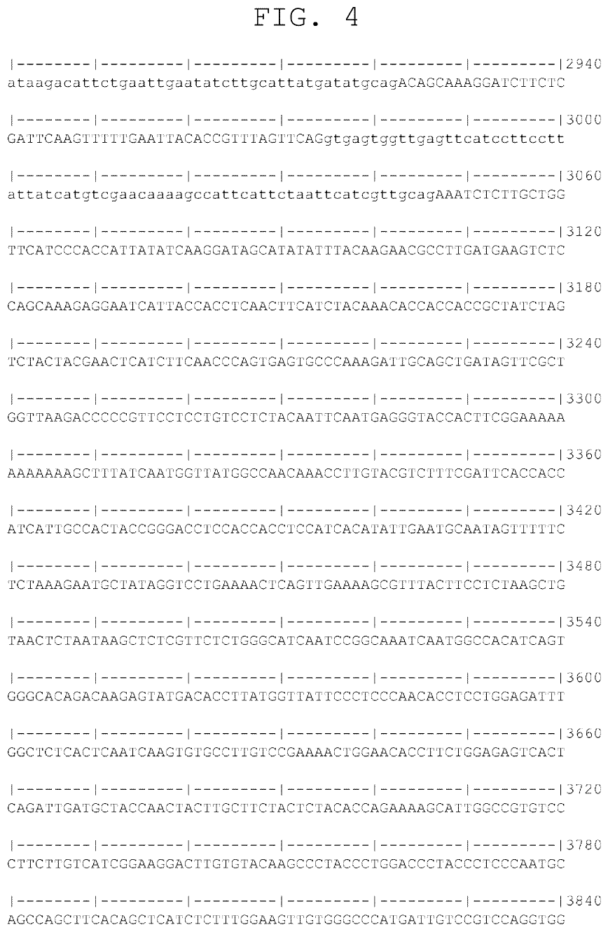

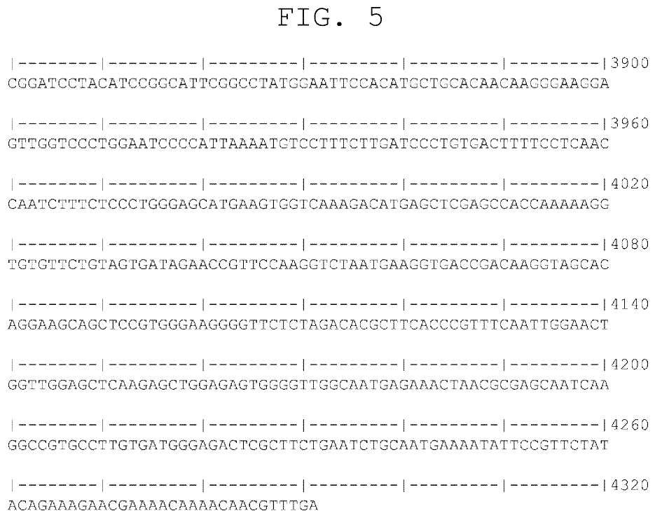

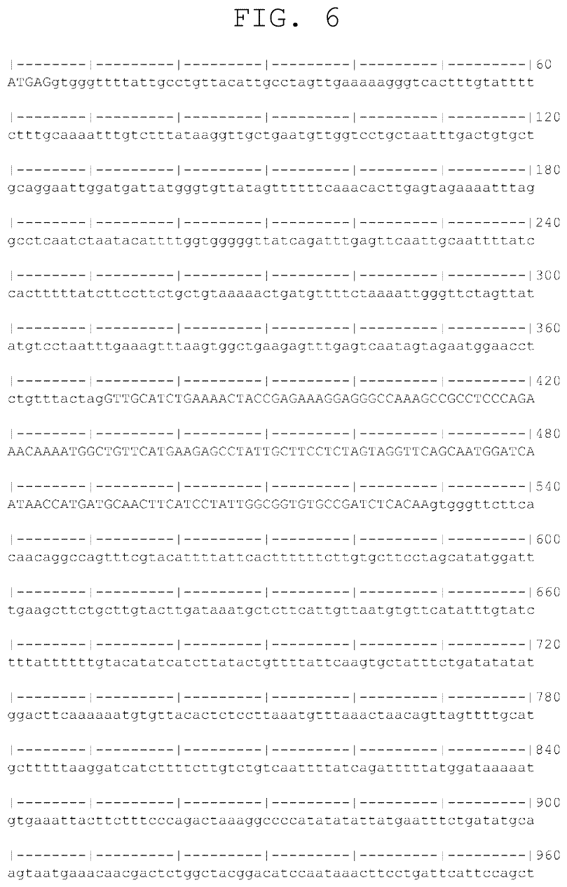

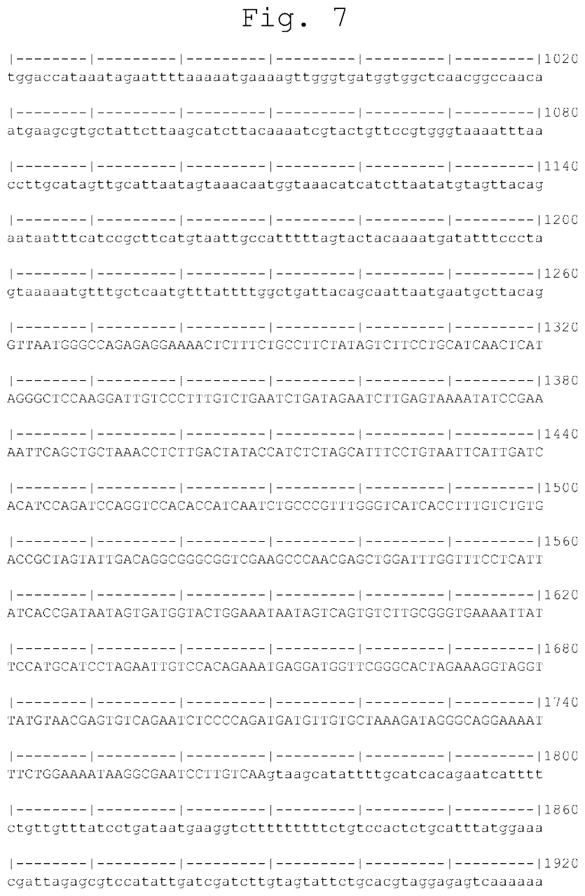

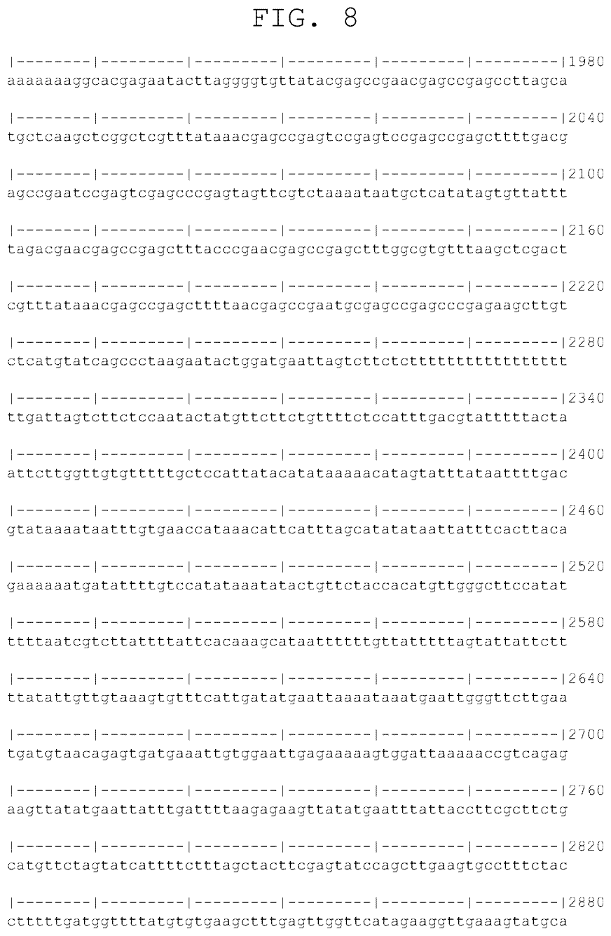

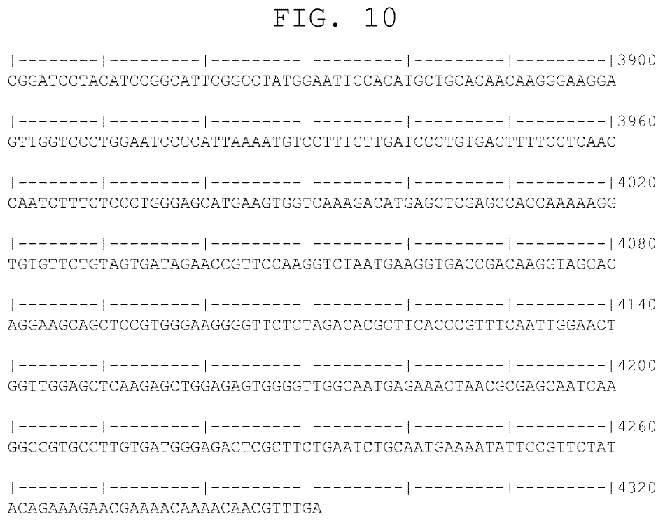

FIGS. 1 to 5 show the genomic nucleotide sequence of the normal S-ELF3 gene, and FIGS. 6 to 10 show the genomic nucleotide sequence of the mutant S-ELF3-PS1 gene produced by the present invention. In FIGS. 1 to 10, the sequences of internal introns are indicated in lower case, and a site where a mutation occurred and a stop codon newly generated because splicing was no longer possible are indicated in boldface and underlines.

Next, an approach of developing a self-propagating Fagopyrum plant (self-propagating buckwheat) using the S-ELF3-PS1 gene will be described.

Development of Self-Propagating Buckwheat

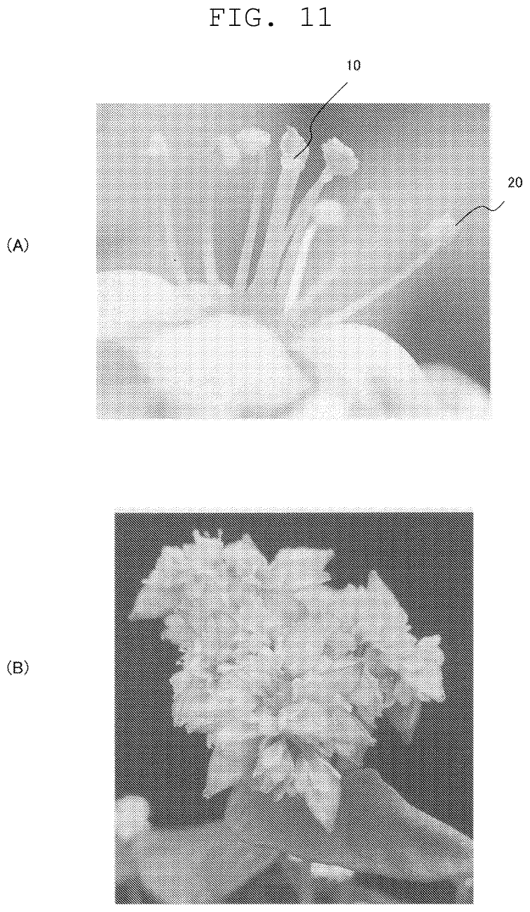

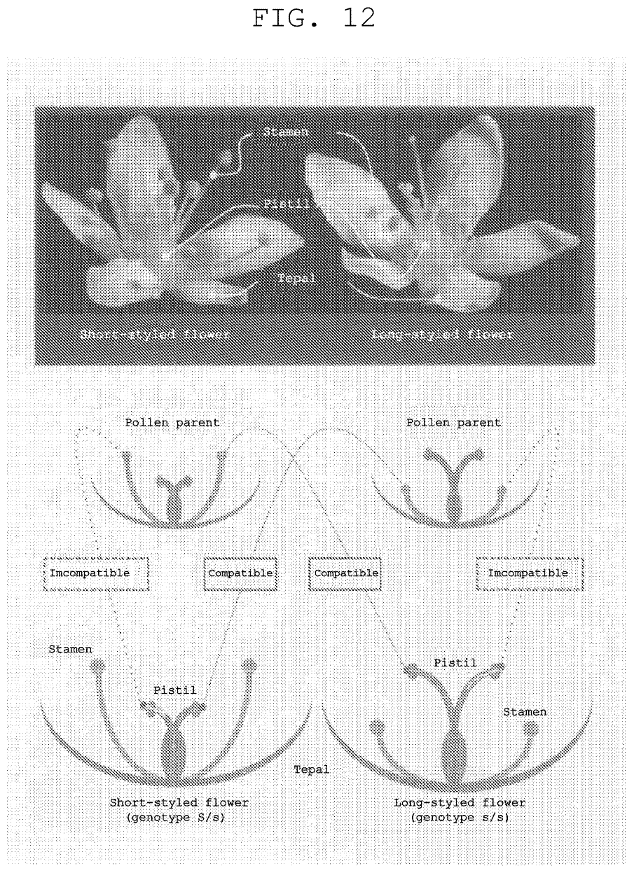

As a result of confirming the flowers of buckwheat individuals having the S-ELF3-PS1 gene, both stamen 20 and pistil 10 were long as shown in FIG. 11. FIG. 11(A) is an enlarged photograph of the flower of a self-propagating Fagopyrum plant retaining the S-ELF3-PS1 gene, and FIG. 11(B) shows a manner in which the flower of the self-propagating Fagopyrum plant retaining the S-ELF3-PS1 gene self-propagates actively.

This individual further produced self-propagating seeds. This individual has S haplotype and is therefore supposed to be a short-styled flower individual. However, because of having the S-ELF3-PS1 gene, pistil became long, and self-propagation was considered to occur. This suggests that the S-ELF3-PS1 gene converted both of traits, pistil length and self-incompatibility, of the short-styled flower to the pistil of a long-styled flower.

When self-propagating individuals heterozygously having the S-ELF3-PS1 gene and having long stamen and pistil (genotype: S-ELF3-PS1/s) were mated with self-incompatible long-styled flower individuals (genotype: s/s), 13 self-propagating individuals having long stamen and pistil, and 28 long-styled flower individuals were obtained in the next generation. All the 13 self-propagating individuals having long stamen and pistil had S-ELF3-PS1, whereas all the 28 self-incompatible long-styled flower individuals had no S-ELF3-PS1.

Thus, self-propagating buckwheat was able to be developed by retaining the S-ELF3-PS1 gene. For the determination of the genotypes, the PCR amplification of the S-ELF3-PS1 gene under the conditions described above was used. The mutation site of the amplified S-ELF3-PS1 gene was confirmed by the Sanger method. The following primer was used in the Sanger method.

S-ELF3_Fw3004_seq,
GCAAAGGATCTTCTCGATTCA

The present invention is not limited by Examples mentioned above, and many changes or modifications can be made in the present invention by the ordinary creativity of those skilled in the art without departing from the technical brief of the present invention.

### REFERENCE SIGNS LIST

10 . . . pistil
20 . . . stamen

1. A loss-of-function transcription factor-like gene produced by:
identifying a transcription factor-like gene formed by a linked gene group that controls lengths of stamen and pistil of a Fagopyrum plant from mapping results of a genomic sequence of the Fagopyrum plant; and
allowing the transcription factor-like gene thus identified to lose a function by mutagenesis.

2. The loss-of-function transcription factor-like gene according to claim 1, wherein
the transcription factor-like gene is
S-ELF3 gene which is present only in a short-styled flower individual having S haplotype and absent in a long-styled flower individual having no S haplotype.

3. The loss-of-function transcription factor-like gene according to claim 1, wherein
the mutagenesis
produces S-ELF3-PS1 gene by allowing the transcription factor-like gene to lose a function by ethyl methanesulfonate treatment.

4. The loss-of-function transcription factor-like gene according to claim 3, wherein
in the S-ELF3-PS1 gene,
a splicing site of intron 3 corresponding to a base at position 3046 of a genomic sequence of the S-ELF3 gene has been altered from G to A so that a codon at positions 2976 to 2978 has become a new stop codon.

5. A self-propagating Fagopyrum plant bred by:
producing S-ELF3-PS1 gene which is a loss-of-function transcription factor-like gene by identifying a transcription factor-like gene formed by a linked gene group that controls lengths of stamen and pistil of a Fagopyrum plant from mapping results of a genomic sequence of the Fagopyrum plant, and allowing the transcription factor-like gene to lose a function by mutagenesis; and
mating and cultivating an individual having this S-ELF3-PS1 self-propagation gene.

6. The self-propagating Fagopyrum plant according to claim 5, wherein
in the mating and the cultivation,
the breeding is performed by mating the individual having the S-ELF3-PS1 gene with a long-styled flower individual to separate an individual having long stamen and pistil.


SEQUENCE LISTING (2 sequences)

SEQ ID NO: 1 - DNA, 4291 bp, Fagopyrum esculentum
atgaggtgggttttattgcctgttacattgcctagttgaaaaagggtcactttgtattttctttgcaaaatttgtctttataaggttgctgaatgttggtcctgctaatttgactgtgctgcaggaattggatgattatgggtgttatagttttttcaaacacttgagtagaaaatttaggcctcaatctaatacattttggtgggggttatcagatttgagttcaattgcaattttatccactttttatcttccttctgctgtaaaaactgatgttttctaaaattgggttctagttatatgtcctaatttgaaagtttaagtggctgaagagtttgagtcaatagtagaatggaacctctgtttactaggttgcatctgaaaactaccgagaaaggagggccaaagccgcctcccagaaacaaaatggctgttcatgaagagcctattgcttcctctagtaggttcagcaatggatcaataaccatgatgcaacttcatcctattggcggtgtgccgatctcacaagtgggttcttcacaacaggccagtttcgtacattttattcacttttttcttgtgcttcctagcatatggatttgaagcttctgcttgtacttgataaatgctcttcattgttaatgtgttcatatttgtatctttattttttgtacatatcatcttatactgttttattcaagtgctatttctgatatatatggacttcaaaaaatgtgttacactctccttaaatgtttaaactaacagttagttttgcatgctttttaaggatcatcttttcttgtctgtcaattttatcagatttttatggataaaaatgtgaaattacttctttcccagactaaaggccccatatatattatgaatttctgatatgcaagtaatgaaacaacgactctggctacggacatccaataaacttcctgattcattccagcttggaccataaatagaattttaaaaatgaaaagttgggtgatggtggctcaacggccaacaatgaagcgtgctattcttaagcatcttacaaaatcgtactgttccgtgggtaaaatttaaccttgcatagttgcattaatagtaaacaatggtaaacatcatcttaatatgtagttacagaataatttcatccgcttcatgtaattgccatttttagtactacaaaatgatatttccctagtaaaaatgtttgctcaatgtttattttggctgattacagcaattaatgaatgcttacaggttaatgggccagagaggaaaactctttctgccttctatagtcttcctgcatcaactcatagggctccaaggattgtccctttgtctgaatctgatagaatcttgagtaaaatatccgaaaattcagctgctaaacctcttgactataccatctctagcatttcctgtaattcattgatcacatccagatccaggtccacaccatcaatctgcccgtttgggtcatcacctttgtctgtgaccgctagtattgacaggcgggcggtcgaagcccaacgagctggatttggtttcctcattatcaccgataatagtgatggtactggaaataatagtcagtgtcttgcgggtgaaaattattccatgcatcctagaattgtccacagaaatgaggatggttcgggcactagaaaggtaggttatgtaacgagtgtcagaatctccccagatgatgttgtgctaaagatagggcaggaaaatttctggaaaataaggcgaatccttgtcaagtaagcatattttgcatcacagaatcattttctgttgtttatcctgataatgaaggtctttttttttctgtccactctgcatttatggaaacgattagagcgtccatattgatcgatcttgtagtattctgcacgtaggagagtcaaaaaaaaaaaaaggcacgagaatacttaggggtgttatacgagccgaacgagccgagccttagcatgctcaagctcggctcgtttataaacgagccgagtccgagtccgagccgagcttttgacgagccgaatccgagtcgagcccgagtagttcgtctaaaataatgctcatatagtgttattttagacgaacgagccgagctttacccgaacgagccgagctttggcgtgtttaagctcgactcgtttataaacgagccgagcttttaacgagccgaatgcgagccgagcccgagaagcttgtctcatgtatcagccctaagaatactggatgaattagtcttctctttttttttttttttttttgattagtcttctccaatactatgttcttctgttttctccatttgacgtatttttactaattcttggttgtgtttttgctccattatacatataaaaacatagtatttataattttgacgtataaaataatttgtgaaccataaacattcatttagcatatataattatttcacttacagaaaaaatgatattttgtccatataaatatactgttctaccacatgttgggcttccatatttttaatcgtcttattttattcacaaagcataattttttgttatttttagtattattcttttatattgttgtaaagtgtttcattgatatgaattaaaataaatgaattgggttcttgaatgatgtaacagagtgatgaaattgtggaattgagaaaaagtggattaaaaaccgtcagagaagttatatgaattatttgattttaagagaagttatatgaatttattaccttcgcttctgcatgttctagtatcattttctttagctacttcgagtatccagcttgaagtgcctttctacctttttgatggttttatgtgtgaagctttgagttggttcatagaaggttgaaagtatgcaataagacattctgaattgaatatcttgcattatgatatgcagacagcaaaggatcttctcgattcaagtttttgaattacaccgtttagttcaggtgagtggttgagttcatccttccttattatcatgtcgaacaaaagccattcattctaattcatcgttgcagaaatctcttgctggttcatcccaccattatatcaaggatagcatatatttacaagaacgccttgatgaagtctccagcaaagaggaatcattaccacctcaacttcatctacaaacaccaccaccgctatctagtctactacgaactcatcttcaacccagtgagtgcccaaagattgcagctgatagttcgctggttaagacccccgttcctcctgtcctctacaattcaatgagggtaccacttcggaaaaaaaaaaaagctttatcaatggttatggccaacaaaccttgtacgtctttcgattcaccaccatcattgccactaccgggacctccaccacctccatcacatattgaatgcaatagtttttctctaaagaatgctataggtcctgaaaactcagttgaaaagcgtttacttcctctaagctgtaactctaataagctctcgttctctgggcatcaatccggcaaatcaatggccacatcagtgggcacagacaagagtatgacaccttatggttattccctcccaacacctcctggagatttggctctcactcaatcaagtgtgccttgtccgaaaactggaacaccttctggagagtcactcagattgatgctaccaactacttgcttctactctacaccagaaaagcattggccgtgtcccttcttgtcatcggaaggacttgtgtacaagccctaccctggaccctaccctcccaatgcagccagcttcacagctcatctctttggaagttgtgggcccatgattgtccgtccaggtggcggatcctacatccggcattcggcctatggaattccacatgctgcacaacaagggaaggagttggtccctggaatccccattaaaatgtcctttcttgatccctgtgacttttcctcaaccaatctttctccctgggagcatgaagtggtcaaagacatgagctcgagccaccaaaaaggtgtgttctgtagtgatagaaccgttccaaggtctaatgaaggtgaccgacaaggtagcacaggaagcagctccgtgggaaggggttctctagacacgcttcacccgtttcaattggaactggttggagctcaagagctggagagtggggttggcaatgagaaactaacgcgagcaatcaaggccgtgccttgtgatgggagactcgcttctgaatctgcaatgaaaatattccgttctatacagaaagaacgaaaacaaaacaacgtttga

SEQ ID NO: 2 - DNA, 4291 bp, Fagopyrum esculentum
atgaggtgggttttattgcctgttacattgcctagttgaaaaagggtcactttgtattttctttgcaaaatttgtctttataaggttgctgaatgttggtcctgctaatttgactgtgctgcaggaattggatgattatgggtgttatagttttttcaaacacttgagtagaaaatttaggcctcaatctaatacattttggtgggggttatcagatttgagttcaattgcaattttatccactttttatcttccttctgctgtaaaaactgatgttttctaaaattgggttctagttatatgtcctaatttgaaagtttaagtggctgaagagtttgagtcaatagtagaatggaacctctgtttactaggttgcatctgaaaactaccgagaaaggagggccaaagccgcctcccagaaacaaaatggctgttcatgaagagcctattgcttcctctagtaggttcagcaatggatcaataaccatgatgcaacttcatcctattggcggtgtgccgatctcacaagtgggttcttcacaacaggccagtttcgtacattttattcacttttttcttgtgcttcctagcatatggatttgaagcttctgcttgtacttgataaatgctcttcattgttaatgtgttcatatttgtatctttattttttgtacatatcatcttatactgttttattcaagtgctatttctgatatatatggacttcaaaaaatgtgttacactctccttaaatgtttaaactaacagttagttttgcatgctttttaaggatcatcttttcttgtctgtcaattttatcagatttttatggataaaaatgtgaaattacttctttcccagactaaaggccccatatatattatgaatttctgatatgcaagtaatgaaacaacgactctggctacggacatccaataaacttcctgattcattccagcttggaccataaatagaattttaaaaatgaaaagttgggtgatggtggctcaacggccaacaatgaagcgtgctattcttaagcatcttacaaaatcgtactgttccgtgggtaaaatttaaccttgcatagttgcattaatagtaaacaatggtaaacatcatcttaatatgtagttacagaataatttcatccgcttcatgtaattgccatttttagtactacaaaatgatatttccctagtaaaaatgtttgctcaatgtttattttggctgattacagcaattaatgaatgcttacaggttaatgggccagagaggaaaactctttctgccttctatagtcttcctgcatcaactcatagggctccaaggattgtccctttgtctgaatctgatagaatcttgagtaaaatatccgaaaattcagctgctaaacctcttgactataccatctctagcatttcctgtaattcattgatcacatccagatccaggtccacaccatcaatctgcccgtttgggtcatcacctttgtctgtgaccgctagtattgacaggcgggcggtcgaagcccaacgagctggatttggtttcctcattatcaccgataatagtgatggtactggaaataatagtcagtgtcttgcgggtgaaaattattccatgcatcctagaattgtccacagaaatgaggatggttcgggcactagaaaggtaggttatgtaacgagtgtcagaatctccccagatgatgttgtgctaaagatagggcaggaaaatttctggaaaataaggcgaatccttgtcaagtaagcatattttgcatcacagaatcattttctgttgtttatcctgataatgaaggtctttttttttctgtccactctgcatttatggaaacgattagagcgtccatattgatcgatcttgtagtattctgcacgtaggagagtcaaaaaaaaaaaaaggcacgagaatacttaggggtgttatacgagccgaacgagccgagccttagcatgctcaagctcggctcgtttataaacgagccgagtccgagtccgagccgagcttttgacgagccgaatccgagtcgagcccgagtagttcgtctaaaataatgctcatatagtgttattttagacgaacgagccgagctttacccgaacgagccgagctttggcgtgtttaagctcgactcgtttataaacgagccgagcttttaacgagccgaatgcgagccgagcccgagaagcttgtctcatgtatcagccctaagaatactggatgaattagtcttctctttttttttttttttttttgattagtcttctccaatactatgttcttctgttttctccatttgacgtatttttactaattcttggttgtgtttttgctccattatacatataaaaacatagtatttataattttgacgtataaaataatttgtgaaccataaacattcatttagcatatataattatttcacttacagaaaaaatgatattttgtccatataaatatactgttctaccacatgttgggcttccatatttttaatcgtcttattttattcacaaagcataattttttgttatttttagtattattcttttatattgttgtaaagtgtttcattgatatgaattaaaataaatgaattgggttcttgaatgatgtaacagagtgatgaaattgtggaattgagaaaaagtggattaaaaaccgtcagagaagttatatgaattatttgattttaagagaagttatatgaatttattaccttcgcttctgcatgttctagtatcattttctttagctacttcgagtatccagcttgaagtgcctttctacctttttgatggttttatgtgtgaagctttgagttggttcatagaaggttgaaagtatgcaataagacattctgaattgaatatcttgcattatgatatgcagacagcaaaggatcttctcgattcaagtttttgaattacaccgtttagttcaggtgagtggttgagttcatccttccttattatcatgtcgaacaaaagccattcattctaattcatcgttgcaaaaatctcttgctggttcatcccaccattatatcaaggatagcatatatttacaagaacgccttgatgaagtctccagcaaagaggaatcattaccacctcaacttcatctacaaacaccaccaccgctatctagtctactacgaactcatcttcaacccagtgagtgcccaaagattgcagctgatagttcgctggttaagacccccgttcctcctgtcctctacaattcaatgagggtaccacttcggaaaaaaaaaaaagctttatcaatggttatggccaacaaaccttgtacgtctttcgattcaccaccatcattgccactaccgggacctccaccacctccatcacatattgaatgcaatagtttttctctaaagaatgctataggtcctgaaaactcagttgaaaagcgtttacttcctctaagctgtaactctaataagctctcgttctctgggcatcaatccggcaaatcaatggccacatcagtgggcacagacaagagtatgacaccttatggttattccctcccaacacctcctggagatttggctctcactcaatcaagtgtgccttgtccgaaaactggaacaccttctggagagtcactcagattgatgctaccaactacttgcttctactctacaccagaaaagcattggccgtgtcccttcttgtcatcggaaggacttgtgtacaagccctaccctggaccctaccctcccaatgcagccagcttcacagctcatctctttggaagttgtgggcccatgattgtccgtccaggtggcggatcctacatccggcattcggcctatggaattccacatgctgcacaacaagggaaggagttggtccctggaatccccattaaaatgtcctttcttgatccctgtgacttttcctcaaccaatctttctccctgggagcatgaagtggtcaaagacatgagctcgagccaccaaaaaggtgtgttctgtagtgatagaaccgttccaaggtctaatgaaggtgaccgacaaggtagcacaggaagcagctccgtgggaaggggttctctagacacgcttcacccgtttcaattggaactggttggagctcaagagctggagagtggggttggcaatgagaaactaacgcgagcaatcaaggccgtgccttgtgatgggagactcgcttctgaatctgcaatgaaaatattccgttctatacagaaagaacgaaaacaaaacaacgtttga